(19) United States
(12) Patent Application Publication
(10) Pub. No.: US 2024/0261211 A1
(43) Pub. Date: Aug. 8, 2024

(54) METHOD FOR PREPARING FILLER COMPOSITION FOR MOLDING

(71) Applicant: ULTRA V CO., LTD., Incheon (KR)

(72) Inventors: Han Jin KWON, Seoul (KR); Jung Ryul HAM, Paju-si (KR); Won Ku LEE, Gimpo-si (KR); Yeon Ju GU, Seoul (KR)

(73) Assignee: ULTRA V CO., LTD., Incheon (KR)

(21) Appl. No.: 18/432,686

(22) Filed: Feb. 5, 2024

(30) Foreign Application Priority Data
Feb. 6, 2023 (KR) 10-2023-0015407

(51) Int. Cl. A61K 8/85; A61K 8/02; A61Q 19/08; C08J 7/12

(52) U.S. Cl. CPC A61K 8/85; A61K 8/0241; A61Q 19/08; C08J 7/123; A61K 2800/412; A61K 2800/654; A61K 2800/91; C08J 2367/04; C08J 2467/04

(57) ABSTRACT

The present disclosure relates to a method for preparing a filler composition for molding. The method for preparing a filler composition for molding includes the operations of: a) providing PLGA microparticles consisting of poly (lactic-co-glycolic acid) (PLGA), a biodegradable polymer; b) providing PDO microparticles consisting of polydioxanone (PDO); c) preparing a mixture by mixing the PLGA microparticles and the PDO microparticles; d) injecting plasma into the mixture to perform a plasma surface treatment on the PLGA microparticles and the PDO microparticles; e) dispersing the plasma-treated mixture into a solution containing a dispersant composition to prepare a dispersant solution; and f) freeze-drying the dispersant solution to form the composite containing the PLGA microparticles and the PDO microparticles.

### CROSS-REFERENCE TO RELATED APPLICATION

A claim for priority under 35 U.S.C. § 119 is made to Korean Patent Application No. 10-2023-0015407 on Feb. 6, 2023. The disclosures of the above-listed applications are hereby incorporated by reference herein in their entirety.

### BACKGROUND

### 1. Technical Field

The present disclosure relates to a method for preparing a filler composition for molding, comprising a mixture of biodegradable polymer microparticles, wherein PLGA microparticles and PDO microparticles are mixed to produce a microparticle mixture, and the PLGA microparticles are provided to constitute 50% to 90% of the total weight of the mixture to enhance cell adhesion and promote extrinsic factor response, thereby maintaining the generated collagen for a long period of time.

### 2. Description of Related Art

The aging population in the anti-aging industry, such as skincare, functional cosmetics, fillers, botulinum toxin, and beauty services, is expanding among the middle class and the general population. With the extension of lifespans, wellness trends, and the advancement of biotechnology, interest in anti-aging is increasing throughout society.

The anti-aging market can be divided into the consumer goods field such as cosmetics, the medical field, and the service field. The medical field is growing with the development of related medicines, such as botulinum toxin and hyaluronic acid fillers, and biomaterials. In the case of botulinum toxin, originally used for treatment of squinting, due to a muscle relaxation effect thereof, in medical institutions in Korea, about 90% of botulinum toxin is used for cosmetic purposes, and with the commercialization of safe and rapidly absorbable pharmaceutical materials such as hyaluronic acid and collagen, the filler market is also growing rapidly.

### RELATED ART

### Patent Documents

Patent Document 1: KR Patent No. 10-1854540

### SUMMARY

An aspect of the present disclosure relates to method for preparing a filler composition for molding. Among fillers for molding, microparticles formed of hydrophobic biodegradable polymers are excreted in urine by being completely decomposed by hydrolysis in the body, and are adjustable for various periods ranging from as short as 6 months to as long as 4 years, depending on the type and molecular weight of the polymer.

Various filler compositions composed of hydrophobic biodegradable polymers, such as poly-L-lactic acid (PLLA), poly-ε-caprolactone (PCL), poly (lactic-co-glycolic acid) (PLGA), and polydioxanone (PDO), have been proposed.

However, PDO has a disadvantage of being difficult to maintain for a long period of time due to the short decomposition period thereof. The decomposition period of PLLA, PCL, and PLGA can be maintained for over a year. However, since PLLA, PCL, and PLGA have higher hydrophobicity than PDO, the side effect, such as granuloma, is frequently reported.

Specifically, after hydrophobic biodegradable polymer microparticles are injected into the human body, collagen can be regenerated through a foreign body reaction. However, in the above process, since cell adhesion to the surface of hydrophobic biodegradable polymer microparticles is difficult, collagen regeneration may be slow or inadequate. Therefore, fillers, which are favorable for cell adhesion, and have a maintenance period of over one year within the body are required.

The present disclosure has been made to solve the above-mentioned problems occurring in the prior art, and in an aspect of the present disclosure, an object of the present disclosure is to provide a method for preparing a filler composition comprising a mixture of biodegradable polymer microparticles. Specifically, PLGA microparticles and PDO microparticles are mixed to produce a microparticle mixture, and the PLGA microparticles are manufactured to constitute 50% to 90% of the total weight of the mixture to enhance cell adhesion and promote extrinsic factor response, thereby maintaining the generated collagen for a long period of time.

The aspects and objectives of the present disclosure are not limited to those mentioned above, and other aspects and objectives not mentioned herein will be clearly understood by those skilled in the art from the following description.

To accomplish the above-mentioned objects, according to an aspect of the present disclosure, there is provided a method for preparing a filler composition including the operations of: a) providing PLGA microparticles consisting of poly (lactic-co-glycolic acid) (PLGA), a biodegradable polymer; b) providing PDO microparticles consisting of polydioxanone (PDO); c) preparing a mixture by mixing the PLGA microparticles and the PDO microparticles; d) injecting plasma into the mixture to perform a plasma surface treatment on the PLGA microparticles and the PDO microparticles; e) dispersing the plasma-treated mixture into a solution containing a dispersant composition to prepare a dispersant solution; and f) freeze-drying the dispersant solution to form the composite containing the PLGA microparticles and the PDO microparticles.

The dispersing agent of the present disclosure is used to disperse physiological active substances, and can be a low molecular weight dispersant polymer or a high molecular weight dispersant polymer. The low molecular weight dispersant refers to a compound with an average molecular weight (Mw) of less than 15,000, and the high molecular weight dispersant refers to a compound including repetitive covalent bonds between one or more monomers, with an average molecular weight (Mw) of 15,000 or more. There are no specific limitations in the low molecular weight dispersant if the dispersant is allowed for pharmaceutical compounds, functional food compounds, functional cosmetic compounds, etc. Specifically, sugars, cyclodextrins, amino acids, organic acids, and other components can be used alone or as combination of two or more types thereof.

The dispersant may be a cell or a growth factor. The cell or the growth factor may be any one selected from the groups consisting of mesenchymal stem cells, induced pluripotent stem cells (iPSC), fibroblast growth factor (FGF), epidermal growth factor (EGF), keratinocyte growth factor (KGF), transforming growth factor-alpha (TGF-α), transforming growth factor-beta (TGF-β), granulocyte colony-stimulating factor (GCSF), insulin-like growth factor (IGF), vascular endothelial growth factor (VEGF), hepatocyte growth factor (HGF), platelet-derived growth factor-BB (PDGF-BB), brain-derived neurotrophic factor (BDNF), and glial cell-derived neurotrophic factor (GDNF).

The dispersant may be amino acid, peptide, or protein. Amino acids, peptides, and proteins that can be used include alanine, arginine, asparagine, aspartic acid, cysteine, glutamine, glutamic acid, glycine, histidine, isoleucine, lysine, methionine, phenylalanine, proline, serine, threonine, tryptophan, tyrosine, valine, or combinations thereof.

The dispersant may be carbohydrate. Carbohydrate may include glucose, mannose, idose, galactose, fucose, ribose, xylose, lactose, sucrose, maltose, trehalose, turanose, raffinose, maltotriose, acarbose, water-soluble cellulose, synthetic cellulose, sugar alcohol, glycerin, sorbitol, lactitol, maltitol, mannitol, xylitol, erythritol, polyol, or derivatives thereof.

The dispersant may be a natural polymer. The natural polymer may include biocompatible natural polymer such as hyaluronic acid, chitosan, alginate, or combinations thereof.

In the method for preparing a filler composition for molding according to an embodiment of the present invention, the weight ratio of the PLGA microparticles to the total weight of the mixture ranges from 50% to 90%.

In the method for preparing a filler composition for molding according to an embodiment of the present invention, the weight ratio of the PDO microparticles to the total weight of the mixture ranges from 10% to 50%.

In the method for preparing a filler composition for molding according to an embodiment of the present invention, in operation f), the dispersant is freeze-dried at a temperature of −80° ° C. to −70° C. for 10 hours to 20 hours.

In the method for preparing a filler composition for molding according to an embodiment of the present invention, the plasma surface treatment in operation d) is performed by injecting air, argon, oxygen or helium gas at a rate of 1 cc/min to 20 cc/min and applying a voltage of 50 W to 110 W to the mixture to inject plasma-treated air, argon, oxygen or helium gas into the mixture for 20 minutes to 40 minutes.

In operation a), the operation of providing PLGA microparticles includes preparing a solution of PLGA dissolved in a solvent to produce a solution 1-1, wherein the solvent is at least one selected from the groups consisting of chloroform, acetone, acetonitrile, anisole, dioxane, methoxybenzene, dichloromethane, dimethylformamide, hexafluoroisopropanol, dimethylsulfoxide, and ethylacetate, or combinations thereof.

The method for preparing a filler composition for molding according to an embodiment of the present invention further includes the operation of: dissolving the solution 1-1 and polyvinyl alcohol (PVA), which is a surfactant, in distilled water to prepare a solution 1-2.

The method for preparing a filler composition for molding according to an embodiment of the present invention further includes the operation of: removing the solvent while mixing and stirring the solution 1-1 and the solution 1-2, to prepare a solution 1-3.

The method for preparing a filler composition for molding according to an embodiment of the present invention further includes the operation of: precipitating the PLGA microparticles in the produced solution 1-3, and removing the supernatant to obtain the PLGA microparticles.

The method for preparing a filler composition for molding according to an embodiment of the present invention further includes the operation of: washing the obtained PLGA microparticles.

In the method for preparing a filler composition for molding according to an embodiment of the present invention, the average size of the PLGA microparticles is from 20 μm to 200 μm.

In operation b), the operation of providing the PDO microparticles includes dissolving the PDO in hexafluoroisopropanol (HFIP) to prepare a solution 2-1.

The method for preparing a filler composition for molding according to an embodiment of the present invention further includes the operation of: dissolving the solution 2-1 and polyvinyl alcohol (PVA), which is a surfactant, in distilled water to prepare a solution 2-2.

The method for preparing a filler composition for molding according to an embodiment of the present invention further includes the operation of: removing the HFIP while mixing and stirring the solution 2-1 and the solution 2-2, to prepare a solution 2-3.

The method for preparing a filler composition for molding according to an embodiment of the present invention further includes the operation of: precipitating the PDO microparticles in the produced solution 2-3, and removing the supernatant to obtain the PDO microparticles.

The method for preparing a filler composition for molding according to an embodiment of the present invention further includes the operation of: washing the obtained PDO microparticles.

In the method for preparing a filler composition for molding according to an embodiment of the present invention, the average size of the PDO microparticles is from 20 μm to 200 μm.

A filler composition for molding according to another embodiment of the present disclosure can be prepared by the method for preparing a filler composition for molding.

An injector for molding according to a further embodiment of the present disclosure can include the filler composition for molding.

### BRIEF DESCRIPTION OF THE DRAWINGS

FIG. 1 is a flowchart illustrating a method for preparing a filler composition for molding according to an embodiment of the present disclosure.

FIG. 2 is a schematic diagram illustrating an arrangement structure between PLGA microparticles and PDO microparticles within the filler composition for molding according to the present disclosure.

FIG. 3 is a scanning electron microscope (SEM) image of the PDO microparticles.

FIG. 4 is a scanning electron microscope (SEM) image of a mixture of the PDO microparticles and the PLGA microparticles.

FIG. 5 is a scanning electron microscope (SEM) image of a mixture of PDO microparticles, PLGA microparticles, and hyaluronic acid.

FIG. 6 is a comparison photograph of the contact angles of the mixture before and after plasma surface treatment.

FIG. 7 is a comparison photograph of Sirius red positive areas for collagen deposition around the filler.

### DETAILED DESCRIPTION

Advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art can fully understand the scope of the present disclosure. Therefore, the present disclosure will be defined only by the scope of the appended claims. Hereinafter, the specific contents for realizing the present disclosure will be described in detail with reference to the accompanying drawings. Regardless of the drawings, the same reference numbers designates the same components, and “and/or” encompasses each item mentioned and one or more combinations thereof.

Terms used in the specification are used to describe specific embodiments of the present disclosure and are not intended to limit the scope of the present disclosure. In the specification, the terms of a singular form may include plural forms unless otherwise specified. It should be also understood that the terms of ‘comprises’ and/or ‘comprising’ in the specification are used to mean that there is no intent to exclude existence or addition of other components besides components described in the specification.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

Hereinafter, a method for preparing a filler composition for molding according to an embodiment of the present disclosure will be described.

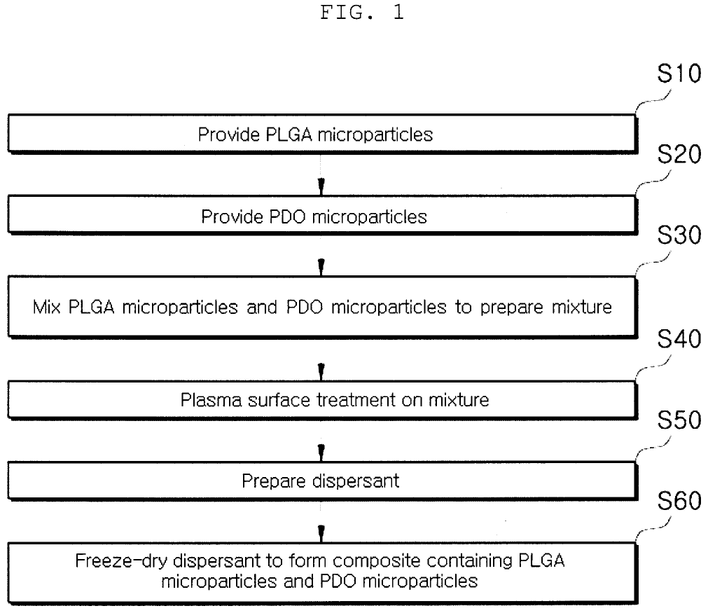

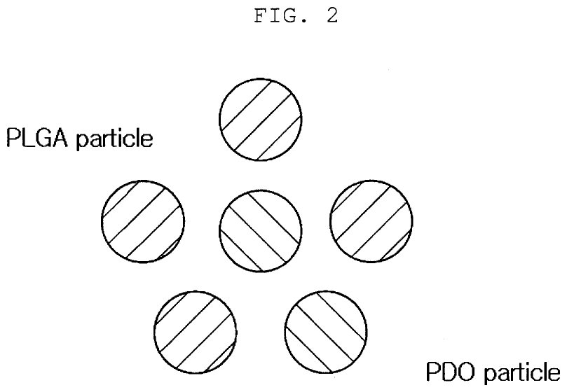

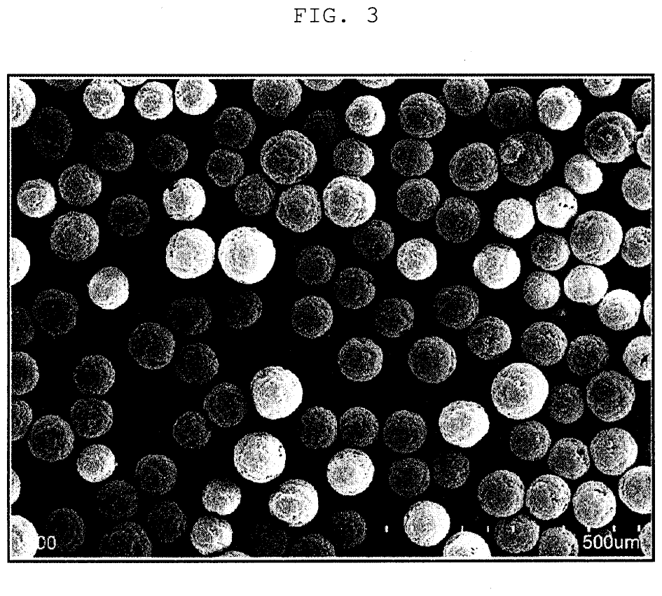

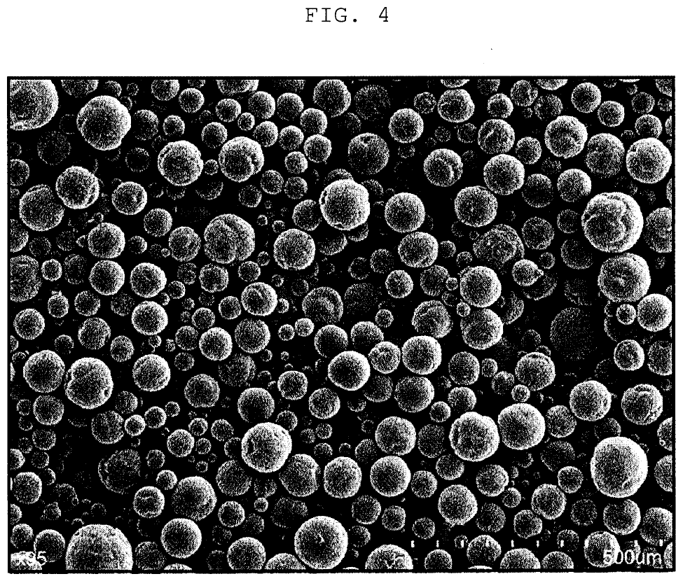

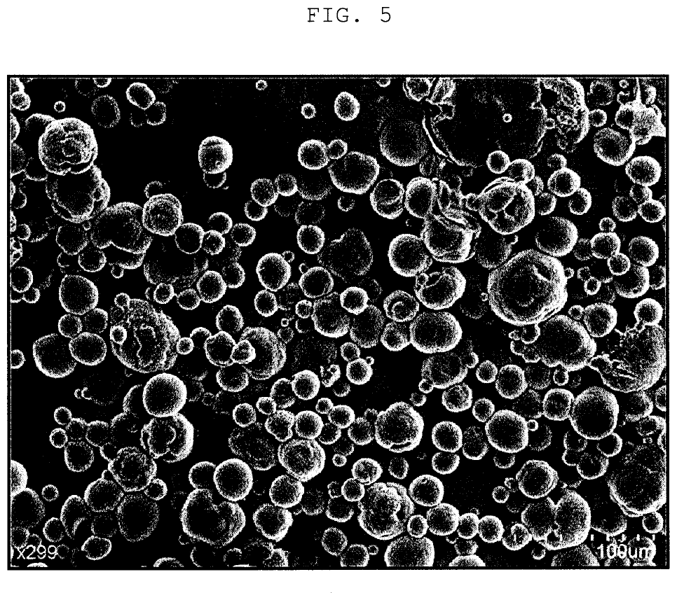

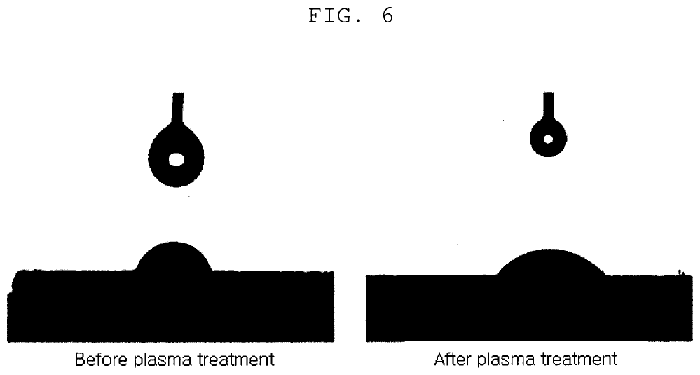

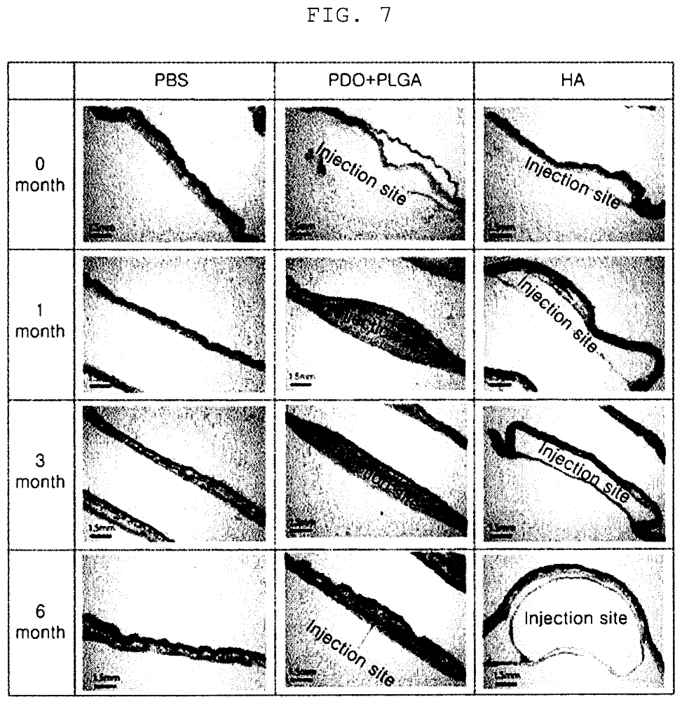

FIG. 1 is a flowchart illustrating a method for preparing a filler composition for molding according to an embodiment of the present disclosure, FIG. 2 is a schematic diagram illustrating an arrangement structure between PLGA microparticles and PDO microparticles within the filler composition for molding according to the present disclosure, FIG. 3 is a scanning electron microscope (SEM) image of the PDO microparticles, FIG. 4 is a scanning electron microscope (SEM) image of a mixture of the PDO microparticles and the PLGA microparticles, FIG. 5 is a scanning electron microscope (SEM) image of a mixture of PDO microparticles, PLGA microparticles, and hyaluronic acid, FIG. 6 is a comparison photograph of the contact angles of the mixture before and after plasma surface treatment, and FIG. 7 is a comparison photograph of Sirius red positive areas for collagen deposition around the filler.

Referring to FIG. 1, the method for preparing a filler composition for molding according to an embodiment of the present disclosure may include: a) providing PLGA microparticles consisting of poly (lactic-co-glycolic acid) (PLGA), a biodegradable polymer; b) providing PDO microparticles consisting of polydioxanone (PDO); c) preparing a mixture by mixing the PLGA microparticles and the PDO microparticles; d) injecting plasma into the mixture to perform a plasma surface treatment on the PLGA microparticles and the PDO microparticles; e) dispersing the plasma-treated mixture into a solution containing a dispersant composition to prepare a dispersant solution; and f) freeze-drying the dispersant solution to the composite form containing the PLGA microparticles and the PDO microparticles.

First, PLGA microparticles are prepared and provided based on poly (lactic-co-glycolic acid) (PLGA), a biodegradable polymer (S10).

The properties of poly (lactic-co-glycolic acid) (PLGA) depend on the composition of glycolide and lactide, but PLGA is excellent in biocompatibility and hydrolyzes into lactic acid and glycolic acid which are harmless in the body, and is maintained without being decomposed for about one to two years, thereby providing excellent durability. PLGA is a material that is not actively hydrolyzed due to the poor water affinity.

To increase hydrophilicity and biodegradability in the body, plasma surface treatment utilizing plasma can be used to increase the decomposition rate within a year. Details regarding plasma surface treatment will be described later.

The weight-average molecular weight of the biodegradable polymer PLGA is 50,000 to 150,000 Da, and the size of microparticles manufactured from biodegradable polymer PLGA may range from 20 to 200 μm. When the size of the microparticles is set within the above range, it is desirable that a residence period in the body of within 24 months can be achieved.

Hydrophobic surfaces like PLGA make it difficult for cells to adhere in the body, so macrophage cannot be adhered onto the surface well, so it prevents collagen generation by the foreign body reaction. On the other hands, a hydrophilic PDO surface is a surface favorable for macrophage adhesion, thereby facilitating collagen generation by the foreign body reaction. (Reference: Kim et al., “The efficacy of powdered polydioxanone in terms of collagen production compared with poly-L-lactic acid in a murine model”, JCD, 2018)

Meanwhile, PLGA microparticles may be provided through the following process.

Specifically, in step a), the step of providing PLGA microparticles includes preparing a solution of PLGA dissolved in a solvent to produce a solution 1-1, wherein the solvent may be at least one selected from the groups consisting of chloroform, acetone, acetonitrile, dioxane, methoxybenzene, dichloromethane, dimethylformamide, dimethylsulfoxide, ethylacetate, hexafluoroisopropanol, and anisole, or combinations thereof. The biodegradable polymer PLGA is dissolved in the solvent to prepare a solution 1-1. The molar ratio of PLA to PGA of PLGA used in one embodiment of the present disclosure ranges from 50:50 to 90:10.

Continuously, polyvinyl alcohol (PVA), which is a surfactant, is dissolved in distilled water to prepare a solution 1-2, and then, the solution 1-1 and the solution 1-2 are mixed together.

Furthermore, while the solution 1-1 the solution 1-2 are mixed and stirred, the solvent of the solution 1-1 is removed to prepare a solution 1-3. Here, the solvent may be at least one selected from the groups consisting of chloroform, acetone, acetonitrile, dioxane, methoxybenzene, dichloromethane, dimethylformamide, dimethylsulfoxide, ethylacetate, hexafluoroisopropanol, and anisole, or combinations thereof.

Additionally, the PLGA microparticles are precipitated in the produced solution 1-3, and the supernatant is removed to obtain the PLGA microparticles. If necessary, operation of washing the obtained PLGA microparticles may be further included. Washing may be performed by adding distilled water to the obtained PLGA microparticles and stirring the mixture. The average size of the produced PLGA microparticles may range from 20 μm to 200 μm.

Next, b) PDO microparticles consisting of polydioxanone (PDO) are provided (S20).

It has been proved that PDO minimizes side effects and is safe due to its biodegradability in the body. However, PDO has a limitation in that the duration of persistence in the body is relatively short, typically up to 6 months, since it is prone to hydrolysis due to hydrophile property.

The weight-average molecular weight of the biodegradable polymer PDO is 50,000 to 150,000 Da, and the size of microparticles manufactured as biodegradable polymer PDO may range from 20 μm to 200 μm. When the size of microparticles is set within the above range, the the duration of persistence in the body can be within 24 months.

Meanwhile, the PDO microparticles may be provided through the following process.

In operation b), the operation of providing the PDO microparticles may include dissolving the PDO in hexafluoroisopropanol (HFIP) to prepare a solution 2-1.

Continuously, polyvinyl alcohol (PVA), which is a surfactant, is dissolved in distilled water to prepare a solution 2-2, and then, the solution 2-1 and the solution 2-2 are mixed together. Thereafter, while the solution 2-1 and the solution 2-2 are mixed and stirred, the HFIP is removed to prepare a solution 2-3.

Additionally, in the produced solution 2-3, the PDO microparticles are precipitated, and the supernatant is removed to obtain the PDO microparticles. If necessary, operation of washing the obtained PDO microparticles may be further included. Washing may be performed by adding distilled water to the obtained PDO microparticles and stirring the mixture. The average size of the produced PDO microparticles may range from 20 μm to 200 μm.

Meanwhile, it is difficult to produce the PDO microparticles in the way similar to the manufacturing process of the PLGA microparticles. This is because PDO is not soluble in representative solvents used to make PLGA microparticles, such as MC and chloroform. Additionally, research papers into producing PDO microparticles have been rarely published except for a few patent documents. Accordingly, it is difficult to produce PDO microparticles. Furthermore, there have been no attempts to utilize the hydrophobic surface properties of PLGA and the relatively hydrophilic surface properties of PDO.

Next, in operation c), the PLGA microparticles and the PDO microparticles are mixed to prepare a mixture (S30). In this instance, the weight ratio of the PLGA microparticles to the total weight of the mixture may range from 50% to 90%. Meanwhile, the weight ratio of the PDO microparticles to the total weight of the mixture may range from 10% to 50%. Outside of the ranges, collagen synthesis is delayed since stable cell adsorption may not occur, and it is difficult to have long-term tissue restoration effects due to the short collagen maintenance period. For example, referring to FIG. 2, in the filler composition including the mixture of the PDO microparticles and the PLGA microparticles, the PDO microparticles are contained at 30% by weight ratio, and the PLGA microparticles are contained up to 70% by weight ratio, so the PDO microparticles can sufficiently exist around the PLGA microparticles.

Next, in operation d), plasma is injected into the mixture to perform a plasma surface treatment on the PLGA microparticles and the PDO microparticles (S40).

Plasma surface treatment can be performed under the following conditions. The plasma surface treatment is performed by injecting air or helium gas at a rate of 1 cc/min to 20 cc/min and applying a voltage of 50 W to 110 W to the mixture to inject plasma-treated air or helium gas into the mixture for 20 minutes to 40 minutes.

Referring to FIG. 6, it can be observed that the water contact angle decreases due to the plasma surface treatment of the mixture. Consequently, as the water contact angle of PLGA microparticles included in the mixture decreases, it can be inferred that the hydrophilic tendency of the PLGA microparticles is more enhanced compared to before the plasma surface treatment. In other words, through the plasma surface treatment, the hydrophobic PLGA microparticles may exhibit a hydrophilic tendency.

Next, in operation e), the plasma-treated mixture is dispersed into a solution containing a dispersing agent to prepare a dispersant (S50).

Next, in operation f), the dispersant is freeze-dried to form a composite including the PLGA microparticles and the PDO microparticles (S60). Here, the composite may take the form of a composite composition including PLGA microparticles, PDO microparticles, and hyaluronic acid.

The freeze-drying of the dispersant can be performed under conditions. That is, the dispersant can be freeze-dried at a temperature of −80° ° C. to −70° C. for 10 hours to 20 hours.

Another embodiment of the present disclosure may provide a filler composition for molding prepared by the method of preparing a filler composition for molding. Furthermore, another embodiment of the present disclosure may provide an injection for molding containing the filler composition for molding.

Herein, the term “injection” may be used in the sense of a substance being injected to the skin area of a subject using a fine needle or a cannular type syringe device.

The injection includes the PLGA microparticles and the PDO microparticles formed of biodegradable polymers of the present disclosure, and may further include a biocompatible carrier. The biocompatible carrier may include one or more selected from alginate and salts thereof, carboxymethyl cellulose and salts thereof, dextran and salts thereof, collagen, gelatin, and elastin. The injection may further include physiologically active substances, local anesthetics, injection water, sterile water, or distilled water, according to the use purposes.

Meanwhile, when the foreign body reaction occurs by the immune system of the human body after the filler is injected into the body, macrophages adhere to the PDO microparticles. At this time, the macrophages naturally adhered to the surface of the PDO microparticles comfortably wrap around the surface of the PLGA microparticles based on the surface of the PDO microparticles.

When only the PLGA microparticles exist, since it is difficult to absorb the macrophages, collagen generation becomes slow or it is difficult to generate collagen. However, when the filler composition for molding of the present disclosure including the mixture is used, it becomes easy to promote collagen generation even on the PLGA microparticles. The filler composition of the present disclosure was injected into hairless mice, and then, changes in skin collagen content and epidermal thickness over time were observed. As results of the observation, it was confirmed that collagen generation was promoted compared to the PBS and HA treatment groups, the generated collagen persisted, and the epidermal thickness increased, thereby showing long-term excellent tissue restoration effects (FIG. 7).

In the filler composition containing the PDO microparticles and the PLGA microparticles, the PDO microparticles are contained at 30% by weight ratio, and the PLGA microparticles are contained at 70% by weight ratio.

Hereinafter, the present disclosure will be described in more detail through embodiments. However, the embodiments are provided for illustrative purposes and do not limit the scope of the present disclosure.

### Embodiment 1: Preparation of PLGA Microparticles

2 g of PLGA (IV: 1.3˜1.7) was dissolved in 50 ml of chloroform to obtain a solution 1-1. 7 g of PVA, a surfactant, was dissolved in 750 ml of distilled water to obtain a solution 1-2. The solution 1-1 and the solution 1-2 were mixed, and the mixture of the solution 1-1 and the solution 1-2 was stirred at 100 rpm to 400 rpm for two days to remove chloroform and obtain a solution 2-3 containing PLGA microparticles. After stirring, the mixture was left as it was for more than 24 hours to precipitate the polymer microparticles, and then, the supernatant was removed and the PLGA microparticles were separated. The separated PLGA microparticles were washed by adding and stirring again distilled water to the separated PLGA microparticles. The washing operation was repeated three times to prepare the PLGA microparticles.

### Embodiment 2: Preparation of PDO Microparticles

2 g of PDO (Inherent viscosity (IV): 1.55 dL/g) was dissolved in 50 ml of hexafluoroisopropanol (HFIP) to prepare a solution 2-1. 2 g of surfactant, polyvinyl alcohol (PVA, Mn 130,000 Da) was dissolved in 50 ml of distilled water to prepare a solution 2-2. The solution 2-1 and the solution 2-2 were mixed, and the mixture was stirred at 400 rpm for three days to remove HFIP and obtain a solution 2-3 containing PDO microparticles. After stirring, the mixture was left as it was for more than 24 hours to precipitate the polymer microparticles, and then, the supernatant was removed and the PDO microparticles were separated. The separated PDO microparticles were washed by adding distilled water to the separated PDO microparticles again and stirring. The washing operation was repeated three times to prepare the PDO microparticles with an average size of 20 to 200 μm (refer to FIG. 3).

### Embodiment 3: Mixing of PLGA Microparticles and PDO Microparticles

Microparticles prepared through Embodiment 1 and Embodiment 2 were mixed according to the composition in Table 1 (refer to FIG. 4).

TABLE 1

| | PDO | PLGA |
|---|---|---|
| Composition | 10 | 90 |
| (%) | 15 | 85 |
| | 20 | 80 |
| | 25 | 75 |
| | 30 | 70 |
| | 40 | 60 |
| | 50 | 50 |

Microparticles were mixed depending on Table 1, so seven types of microparticle mixtures totaling 10 g were prepared. Each mixture was inserted into 100 ml of solution (15%) containing a dispersant composition and stirred for two hours to prepare a completely dispersed solution. The dispersant was poured evenly y into a hemispherical mold (diameter of 1 cm) or a cylindrical container, and was inserted into an ultra-low temperature freezer at −75° C. to completely freeze the dispersant for 12 hours.

Thereafter, freeze-drying was performed to create completely dried composite. Among the composites, the final composite completed by mixing PDO and PLGA in a weight ratio of 15:85 is as shown in FIG. 5.

### Embodiment 4: Plasma Surface Treatment

Plasma was irradiated to the surface of the microparticles of the mixture prepared in Embodiment 3 (mixture containing the PLGA microparticles and the PDO microparticles) through a specially designed plasma generator to enhance hydrophilicity through surface modification of the microparticles. Surface-modified PDO and PLGA microparticles were manufactured by the plasma surface treatment.

Although embodiments of the present disclosure have been described, the present disclosure is not limited to the embodiments disclosed herein and can be manufactured in various different forms. It will be understood by those skilled in the art that the disclosure may be embodied in other concrete forms without changing the technological scope and essential features. Therefore, the above-described embodiments should be considered only as examples in all aspects and not for purposes of limitation.

### Advantageous Effects

The present disclosure has the following effects by the aforementioned configuration. The filler composition for molding composed of mixture of PLGA microparticles and PDO microparticles can reduce the risk of side effects compared to the conventional filler composed of only PLLA, PCL, and PLGA microparticles.

More specifically, the present disclosure can enhance sterilization and hydrophilicity through surface treatment of microparticles using plasma, thereby inducing stable cell adhesion, facilitating collagen generation by the foreign body reaction, and providing prolonged tissue restoration since the PDO microparticles are decomposed and disappear within six months but the PLGA microparticles remain.

1. A method for preparing a filler composition comprising the operations of:
a) providing consisting of PLGA microparticles poly (lactic-co-glycolic acid) (PLGA), a biodegradable polymer;
b) providing PDO microparticles consisting of polydioxanone (PDO);
c) preparing a mixture by mixing the PLGA microparticles and the PDO microparticles;
d) injecting plasma into the mixture to perform a plasma surface treatment on the PLGA microparticles and the PDO microparticles;
e) dispersing the plasma-treated mixture into a solution containing a dispersant composition to prepare a dispersant solution; and
f) freeze-drying the dispersant solution to form the composite containing the PLGA microparticles and the PDO microparticles.

2. The method for preparing a filler composition according to claim 1, wherein the weight ratio of the PLGA microparticles to the total weight of the mixture ranges from 50% to 90%.

3. The method for preparing a filler composition according to claim 1, wherein the weight ratio of the PDO microparticles to the total weight of the mixture ranges from 10% to 50%.

4. The method for preparing a filler composition according to claim 1, wherein the dispersant includes cells, growth factors, amino acids, peptides, proteins, carbohydrates, natural polymers, or combinations thereof.

5. The method for preparing a filler composition according to claim 4, wherein the natural polymers are hyaluronic acid, chitosan, alginate, or combinations thereof.

6. The method for preparing a filler composition according to claim 1, wherein in operation f), the dispersant is freeze-dried at a temperature of −80° ° C. to −70° C. for 10 hours to 20 hours.

7. The method for preparing a filler composition according to claim 1, wherein the plasma surface treatment in operation d) is performed by injecting air or helium gas at a rate of 1 cc/min to 20 cc/min and applying a voltage of 50 W to 110 W to the mixture to inject plasma-treated air or helium gas into the mixture for 20 minutes to 40 minutes.

8. The method for preparing a filler composition according to claim 1, wherein in operation a), the operation of providing PLGA microparticles includes preparing a solution of PLGA dissolved in a solvent to produce a solution 1-1, wherein the solvent is at least one selected from the groups consisting of chloroform, acetone, acetonitrile, dioxane, methoxybenzene, dichloromethane, dimethylformamide, dimethylsulfoxide, ethylacetate, hexafluoroisopropanol, and anisole, or combinations thereof.

9. The method for preparing a filler composition according to claim 8, further comprising the operation of:
dissolving the solution 1-1 and polyvinyl alcohol (PVA), which is a surfactant, in distilled water to prepare a solution 1-2.

10. The method for preparing a filler composition according to claim 8 or 9, further comprising the operation of:
removing the solvent while mixing and stirring the solution 1-1 and the solution 1-2, to prepare a solution 1-3.

11. The method for preparing a filler composition according to claim 10, further comprising the operation of:
precipitating the PLGA microparticles in the produced solution 1-3, and removing the supernatant to obtain the PLGA microparticles.

12. The method for preparing a filler composition according to claim 11, further comprising the operation of:
washing the obtained PLGA microparticles.

13. The method for preparing a filler composition according to claim 11, wherein the average size of the PLGA microparticles is from 20 μm to 200 μm.

14. The method for preparing a filler composition according to claim 1, wherein in operation b), the operation of providing the PDO microparticles includes dissolving the PDO in hexafluoroisopropanol (HFIP) to prepare a solution 2-1.

15. The method for preparing a filler composition according to claim 14, further comprising the operation of:
dissolving the solution 2-1 and polyvinyl alcohol (PVA), which is a surfactant, in distilled water to prepare a solution 2-2.

16. The method for preparing a filler composition according to claim 14 or 15, further comprising the operation of:
removing the HFIP while mixing and stirring the solution 2-1 and the solution 2-2, to prepare a solution 2-3.

17. The method for preparing a filler composition according to claim 16, further comprising the operation of:
precipitating the PDO microparticles in the produced solution 2-3, and removing the supernatant to obtain the PDO microparticles.

18. The method for preparing a filler composition according to claim 17, further comprising the operation of:
washing the obtained PDO microparticles.

19. The method for preparing a filler composition according to claim 17, wherein the average size of the PDO microparticles is from 20 μm to 200 μm.

20. A filler composition for molding prepared by the preparing method of claim 1.

21. An injection for molding containing the filler composition of claim 20.
